(19) United States
(12) Patent Application Publication
(10) Pub. No.: US 2025/0046460 A1
(43) Pub. Date: Feb. 6, 2025

(54) METHOD FOR ESTABLISHING DIGITAL TWIN BASED ON MEDICAL HEALTH, DEVICE, AND STORAGE FOR THE SAME

(71) Applicant: Yunliang WU, Fuzhou (CN)

(72) Inventor: Yunliang WU, Fuzhou (CN)

(21) Appl. No.: 18/926,253

(22) Filed: Oct. 24, 2024

Related U.S. Application Data
(63) Continuation of application No. PCT/CN2023/089809, filed on Apr. 21, 2023.

(30) Foreign Application Priority Data
Apr. 26, 2022 (CN) 202210445206.X

(51) Int. Cl. G16H 50/20; G16H 10/60

(52) U.S. Cl. CPC G16H 50/20; G16H 10/60

(57) ABSTRACT

A method for establishing a digital twin based on medical health, including the following steps: receiving individual user's health data from multiple medical health information systems; generating an individual digital twin model based on the individual user's health data, including a digital representation of carbon-based biochemical data of the human body, a digital representation of perceptual and cognitive data of the human body, and a digital representation of natural entity intervention data; using a machine learning module in the individual digital twin model to perform holistic diagnosis and obtain a digital representation of the user's diagnostic data; using the machine learning module to dynamically match natural entity interventions and obtain a digital representation of the natural entity interventions matching the user; and using the machine learning module to assist in optimizing medical health decision-making, obtaining a digital representation of the optimized medical health decisions matching the user.

### TECHNICAL FIELD

The present invention relates to a method, device, and storage medium for establishing a digital twin based on medical health, belonging to the technical field of digital medical prevention.

### BACKGROUND OF THE INVENTION

The research and application of digital information technologies such as big data, cloud computing, and artificial intelligence in the medical and health field have become a cutting-edge focus. By integrating “Internet+” with medical health, the health industry is experiencing rapid development. Real-world data research is being conducted, deeply integrating medical services and public health foundational information to promote disease prevention and shift towards comprehensive health maintenance and promotion for the entire population. The development of health and medical big data, based on the digitalization of healthcare services, will drive a fundamental shift in the healthcare management model and improve the efficiency of the healthcare system. The practice of precision medicine must necessarily promote the close integration of digital information technologies with medical health.

### Technical Problem

However, the current system and mechanism for the application of health and medical big data are inadequate, and they still cannot effectively promote the development and utilization of big data resources. Issues such as “silos” and “islands” of data are prominent, and medical health data and online health information still urgently need to be integrated. The digital twin of the human body provides direction for solving the digitalization, networking, and intelligentization of healthcare. The human body is a complex macro-system, and how to use digital information technology to establish a parallel interactive system between individual healthcare and health medical big data is the technical problem that urgently needs to be addressed.

### SUMMARY OF THE INVENTION

In order to solve the problems existing in the prior art, the present invention proposes a method, device, and storage medium for establishing a digital twin based on medical health.

The technical solution of the present invention is as follows.

In one aspect, the present invention provides a method for establishing a digital twin based on medical health, comprising the following steps:
receiving health data of individual users from multiple medical health information systems;
generating an individual digital twin model based on the health data of individual users; the individual digital twin model includes a digital representation of the carbon-based biochemical data of the human body, a digital representation of the perceptual and cognitive data of the human body, and a digital representation of natural entity intervention data; the digital representation of the carbon-based biochemical data of the human body characterizes the user's physical physiological features; the digital representation of the perceptual and cognitive data of the human body characterizes the user's psychological perception and cognitive features; the digital representation of the natural entity intervention data characterizes the features of external entity exposure and intervention.

In the individual digital twin model, a machine learning module is also used to perform a holistic diagnosis based on the digital representation of the carbon-based biochemical data of the human body and the digital representation of the perceptual and cognitive data of the human body to obtain a digital representation of the user's diagnostic data; the machine learning module is also used to dynamically match natural entity interventions based on the digital representation of the carbon-based biochemical data of the human body and the digital representation of the natural entity intervention data, obtaining a digital representation of the effects of natural entity interventions matching the user; the machine learning module is also used to assist in optimizing medical health decision-making based on the digital representation of the perceptual and cognitive data of the human body and the digital representation of the natural entity intervention data, obtaining a digital representation of optimized medical health decisions matching the user.

Preferably, the digital representation of the carbon-based biochemical data of the human body includes a digital representation of the microstructure of the body and a digital representation of macroscopic signs; the digital representation of the perceptual and cognitive data of the human body includes at least a digital representation of psychological emotions, a digital representation of habits and preferences, and a digital representation of value orientation; the digital representation of the effects of natural entity interventions includes at least a digital representation of diet, a digital representation of activities, a digital representation of geographical environment, a digital representation of medical product usage, a digital representation of medical health services systems, and a digital representation of data collected by monitoring devices.

Preferably, the step of using a machine learning module to perform a holistic diagnosis based on the digital representation of the carbon-based biochemical data of the human body and the digital representation of the perceptual and cognitive data of the human body to obtain a digital representation of the user's diagnostic data specifically comprises the following steps:
obtaining the digital representation of the carbon-based biochemical data of the human body and the digital representation of the perceptual and cognitive data of the human body from the individual digital twin models of multiple users with health problems, and generating a feature data matrix based on the extracted data;
establishing a recurrent neural network model; inputting the generated feature data matrix, with the corresponding user's health problems as output, to iteratively train the recurrent neural network model; obtaining a diagnostic model;
performing holistic diagnosis using the diagnostic model to obtain the digital representation of the corresponding user's diagnostic data.

Preferably, the step of using the machine learning module to dynamically match natural entity interventions based on the digital representation of the carbon-based biochemical data of the human body and the digital representation of the natural entity intervention data to obtain a digital representation of the effects of natural entity interventions matching the user specifically comprises the following steps:
establishing a machine learning-based prognosis prediction model, using the prognosis prediction model and the individual digital twin model to simulate the impact on the corresponding user after implementing various natural entity interventions;
comparing various natural entity interventions based on the impact on the corresponding user after the implementation of each natural entity intervention; dynamically matching natural entity interventions to the corresponding user based on the comparison results.

Preferably, the method of establishing the machine learning-based prognosis prediction model specifically comprises:
identifying and obtaining multiple users who have implemented a natural entity intervention from multiple medical health information systems;
obtaining the digital representation of the carbon-based biochemical data of the human body and the digital representation of the natural entity intervention data before the implementation of the corresponding natural entity intervention, as well as obtaining the digital representation of the carbon-based biochemical data of the human body and the digital representation of the natural entity intervention data after the implementation of the corresponding natural entity intervention from the individual digital twin models of each user; and establishing an impact label based on the data before and after the implementation of the natural entity intervention; the impact label indicates the effect of the corresponding natural entity intervention on the corresponding user;
establishing a recurrent neural network model, using the digital representation of the carbon-based biochemical data of the human body and the digital representation of the natural entity intervention data before the implementation of the corresponding natural entity intervention as input, and the corresponding impact label as output, to iteratively train the recurrent neural network model, thereby obtaining the prognosis prediction model.

Preferably, the step of using the machine learning module to assist in optimizing medical health decision-making based on the digital representation of the perceptual and cognitive data of the human body and the digital representation of the natural entity intervention data, and obtaining a digital representation of optimized medical health decisions matching the user, specifically comprises the following steps:
identifying and obtaining multiple users who have implemented a natural entity intervention from multiple medical health information systems;
obtaining the digital representation of the perceptual and cognitive data of the human body and the digital representation of the natural entity intervention data before the implementation of the corresponding natural entity intervention from the individual digital twin models of each user; as well as obtaining the digital representation of the carbon-based biochemical data of the human body and the digital representation of the natural entity intervention data after the implementation of the corresponding natural entity intervention; and establishing an evaluation label based on the data before and after the implementation of the natural entity intervention; the evaluation label indicates the corresponding user's assessment of the corresponding natural entity intervention;
establishing a recurrent neural network model; using the digital representation of the perceptual and cognitive data of the human body and the digital representation of the natural entity intervention data before the implementation of the corresponding natural entity intervention as input, and the corresponding evaluation label as output, to iteratively train the recurrent neural network model, thereby obtaining the decision optimization model;
performing holistic diagnosis using the decision optimization model to obtain a digital representation of optimized medical health decisions matching the user.

Preferably, the step of receiving health data of individual users from multiple medical health information systems further comprises:
receiving multiple raw data from multiple medical health information systems for the individual user; calculating at least one new enriched data based on the relationship between the multiple raw data; and placing the multiple raw data and the calculated enriched data into the corresponding user's health data.

In another aspect, the present invention also provides an electronic device, comprising a memory, a processor, and a computer program stored on the memory and executable on the processor, wherein the processor, when executing the program, implements the method for establishing a digital twin based on medical health according to any of the embodiments of the present invention.

In another aspect, the present invention also provides a computer-readable storage medium, on which a computer program is stored, and when the program is executed by a processor, it implements the method for establishing a digital twin based on medical health according to any of the embodiments of the present invention.

In another aspect, the present invention also provides a health management service system for the entire life cycle:

The system comprises a collaborative management module and the individual digital twin model of the user, wherein the individual digital twin model of the user is established by the method for establishing a digital twin based on medical health according to any of the embodiments of the present invention;

The collaborative management module obtains data from the individual digital twin model of the user and provides services to the user based on the obtained data.

### Advantages

The present invention has the following advantages:

The method for establishing a digital twin based on medical health according to the present invention collects multi-source health data and consolidates it into an individual digital twin model composed of a digital representation of the carbon-based biochemical data of the human body, a digital representation of the perceptual and cognitive data of the human body, and a digital representation of natural entity intervention data, enabling the reflection of comprehensive and parallel macro-micro, internal-external, physical-psychological individualized mapping data.

The method for establishing a digital twin based on medical health according to the present invention further generates, through a machine learning module within the individual digital twin model, a digital representation of diagnostic data, a digital representation of natural entity interventions matching the user, and a digital representation of optimized medical health decisions matching the user, thereby assisting in comprehensive and full-cycle precision healthcare.

The method for establishing a digital twin based on medical health according to the present invention also generates enriched data from the multiple raw data received from multiple medical health information systems of the individual user, enhancing the multidimensionality of medical health data.

### BRIEF DESCRIPTION OF DRAWINGS

FIG. 1 is a flowchart of the method according to an embodiment of the present invention;

FIG. 2 is an illustration of the individual digital twin model according to an embodiment of the present invention.

### DETAILED DESCRIPTION OF THE INVENTION

The embodiments of the present invention will be described clearly and completely in conjunction with the accompanying drawings in the embodiments of the present invention. It is evident that the described embodiments are only part of the embodiments of the present invention, rather than all embodiments. Based on the embodiments of the present invention, all other embodiments obtained by those of ordinary skill in the art without any creative efforts shall fall within the scope of protection of the present invention.

It should be understood that the step numbers used herein are merely for convenience of description and do not impose limitations on the sequential execution of the steps.

It should be understood that the terminology used in the present invention is for the purpose of describing specific embodiments only and is not intended to limit the present invention. As used in the present invention and the appended claims, unless otherwise clearly indicated by the context, the singular forms “a,” “an,” and “the” are intended to include the plural forms as well.

The terms “comprises” and “includes” specify the presence of stated features, elements, steps, operations, components, and/or assemblies, but do not preclude the presence or addition of one or more other features, elements, steps, operations, components, and/or assemblies.

The term “and/of” refers to any and all possible combinations of one or more of the associated listed items and combinations thereof.

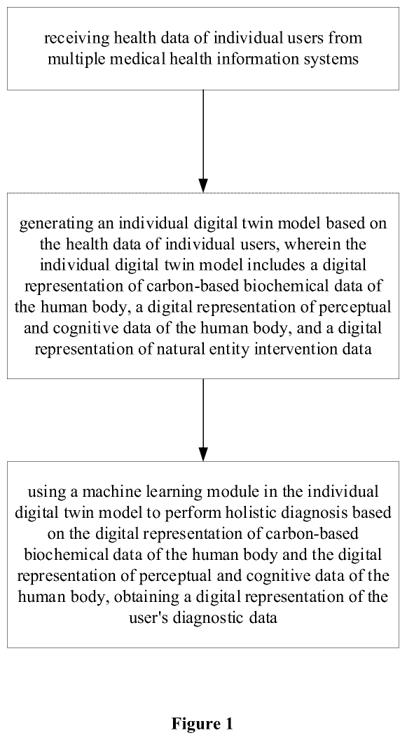

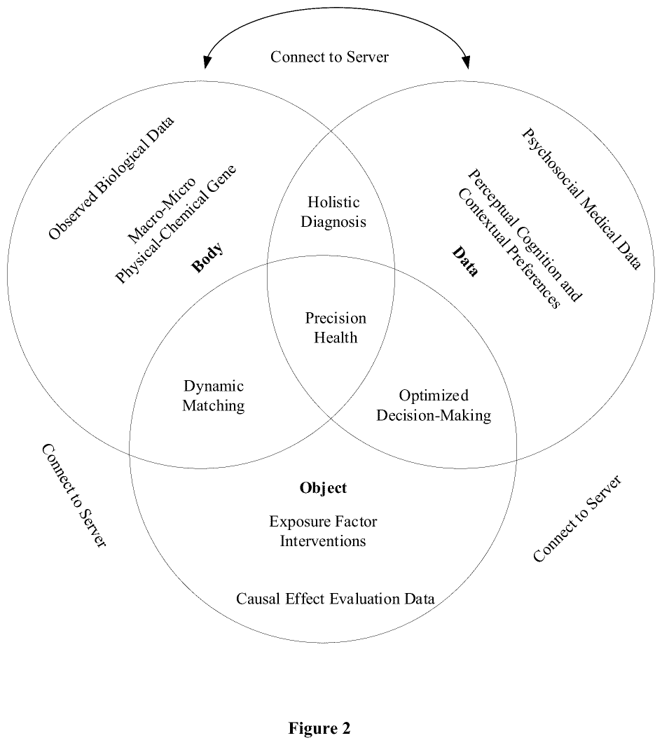

Embodiment 1: Referring to FIG. 1, this embodiment provides a method for establishing a digital twin based on medical health, comprising the following steps:
receiving health data of individual users from multiple medical health information systems; the multiple medical health information systems include data platforms from relevant government departments, data platforms from disease control agencies at all levels, data platforms from medical institutions, data platforms from insurance agencies, data platforms from elderly care institutions, data platforms from pharmaceutical companies, and so on;
generating an individual digital twin model based on the health data of individual users. Referring to FIG. 2, the individual digital twin model generated in this embodiment includes a digital representation of carbon-based biochemical data of the human body, a digital representation of perceptual and cognitive data of the human body, and a digital representation of natural entity intervention data. In this embodiment, the multi-source health data of the individual user is consolidated into an organic whole composed of three key elements-“body, data, and object,” forming an individualized mapping of macro-micro, internal-external, and physical-psychological dimensions.

The “body” (biophysical) refers to the digital representation of carbon-based biochemical data of the human body, which characterizes the user's carbon-based physiological, chemical, and biological data, reflecting biological attributes.

“Data” (informational-physical) refers to the digital representation of perceptual and cognitive data of the human body, which characterizes the user's psychological perception and cognitive features, reflecting social attributes such as scientific understanding, psychology, humanities, economics, etc. Medical means are integrated to assist in perceptual cognition, enabling the acquisition and representation of comprehensive information regarding both the body and external factors as intuitive cognitive data. Psychological perception and cognitive features are composed of the interaction between “self-perception and cognition” and “medical technology-assisted perception and cognition.” The “medical technology-assisted perception and cognition” also includes the digital expression of multidimensional big data analysis and inference results. This medical technology-assisted perception and cognition obtains and represents multidimensional information about the human body and external factors through medical technologies. The medical technology-assisted perception and cognition on multiple scales includes, at a minimum, conventional medicine, traditional Chinese medicine, gene and cellular analysis, and wearable devices.

“Object” (entity-physical) refers to the digital representation of natural entity intervention data, which characterizes the features of external entity interventions, specifically representing full-scenario exposure and intervention factors outside the body, along with evaluation data of these factors.

In the individual digital twin model, a machine learning module is used to perform holistic diagnosis based on the digital representation of the carbon-based biochemical data of the human body and the digital representation of the perceptual and cognitive data of the human body, obtaining a digital representation of the user's diagnostic data. The diagnostic data from the holistic diagnosis reflects not only physical characteristics but also perception, psychology, cognition, environment, society, economy, and preferences. In the individual digital twin model, the machine learning module is also used to dynamically match natural entity interventions based on the digital representation of the carbon-based biochemical data of the human body and the digital representation of natural entity intervention data, obtaining a digital representation of natural entity intervention measures matching the user. The dynamic matching of natural entity interventions includes monitoring and testing to obtain the user's physiological characteristics, exposing the body to natural entity interventions, obtaining the features of natural entity intervention measures, and assessing the effects or impacts of such interventions. In the individual digital twin model, the machine learning module is also used to assist in optimizing medical health decision-making based on the digital representation of the perceptual and cognitive data of the human body and the digital representation of natural entity intervention data, obtaining a digital representation of optimized medical health decisions matching the user. The digital representation of the optimized medical health decisions is updated to the digital representation of medical technology-assisted perceptual cognition, forming an iterative cycle, and is output and stored in the corresponding module of the medical health information system.

In a preferred embodiment of the present embodiment, the digital representation of carbon-based biochemical data of the human body includes a digital representation of the microstructure of the body and a digital representation of macroscopic signs, which at least includes digital representations of physicochemical data, omics data, and observational data. The digital representation of the microstructure of the body includes cellular genes, biochemical molecular minerals, cellular tissue organ systems, and multidimensional spatial structural morphology, while the digital representation of macroscopic signs includes gender, age, and physical morphology.

The digital representation of perceptual and cognitive data of the human body includes a digital representation of psychological emotions, a digital representation of habits and preferences, and a digital representation of value orientation. For example, perceptual psychological data, economic status data, and behavioral habit data. These data are typically self-collected by users through channels such as the Internet and the Internet of Things, and also include data such as nutrition activities, narrative scenarios, and self-intervention.

The digital representation of natural entity intervention data includes a digital representation of diet, a digital representation of activities, a digital representation of geographical environment, a digital representation of medical product usage, a digital representation of medical health services systems, and a digital representation of data collected by monitoring devices. Among them, the digital representation of activities includes at least the exposure or intervention of the human body under the influence of gravity and equipment force, and the medical health services system includes a health services system involving doctors and medical personnel.

Through the data of the individual digital twin model, diagnostic decisions can be made around the optimal matching of “body, data, and object.” Comprehensive and full-cycle precision healthcare can be achieved through longitudinal integration analysis of omics data for individual users, multi-layer systematic analysis of health and medical big data, and other methods. The individual digital twin model generates structured data in formats and content such as “diagnosis results, solutions, detailed measures, and decision recommendations,” which are presented in an intuitive way after processing for usability and visualization.

In a preferred embodiment of the present embodiment, the step of using the machine learning module to perform holistic diagnosis based on the digital representation of the carbon-based biochemical data of the human body and the digital representation of the perceptual and cognitive data of the human body, and obtaining the digital representation of the user's diagnostic data specifically comprises the following steps:
obtaining the digital representation of the carbon-based biochemical data of the human body and the digital representation of the perceptual and cognitive data of the human body from the individual digital twin models of multiple users with health issues; generating a feature data matrix based on the extracted data as training data. In this embodiment, health issues are used as groupings, and the digital representation of the carbon-based biochemical data of the human body and the digital representation of the perceptual and cognitive data of the human body for a large number of users with those health issues are collected under different health issue groups, and the corresponding feature data matrix is generated;
establishing one or more recurrent neural network models. In the case of establishing a recurrent neural network model, the network model is essentially a multi-classification model. The generated feature data matrices are input as training samples into the recurrent neural network model, with the corresponding user's health issues as the output layer of the recurrent neural network model. The recurrent neural network model is iteratively trained to obtain a diagnostic model capable of diagnosing multiple health issues. In the case of establishing multiple recurrent neural network models, one recurrent neural network model may correspond to one health issue, or one recurrent neural network model may correspond to a small number of different health issues. Using the training steps described above, a collection of diagnostic models capable of diagnosing different health issues can be obtained;
using the generated one or more diagnostic models to perform holistic diagnosis, obtaining the digital representation of the corresponding user's diagnostic data. In this embodiment, the digital representation of the diagnostic data specifically refers to the user's risk of suffering from different health issues.

In a preferred embodiment of the present embodiment, the step of using the machine learning module to dynamically match natural entity interventions based on the digital representation of the carbon-based biochemical data of the human body and the digital representation of natural entity intervention data, and obtaining a digital representation of natural entity interventions matching the user, specifically comprises the following steps:
first, determining whether to recommend one or more different natural entity interventions (such as drug treatments, prescriptions, dietary therapies, etc.); inputting this set of natural entity interventions into the machine learning model, which is trained to determine recommendations based on the data from an individual user's digital twin model; obtaining the recommended natural entity interventions and the corresponding confidence scores from the machine learning model based on the data;
establishing a machine learning-based prognosis prediction model, using the prognosis prediction model and the individual digital twin model to simulate the impact on the corresponding user after implementing the recommended natural entity interventions;
comparing the various natural entity interventions based on the impact on the corresponding user after implementation; dynamically matching natural entity interventions to the corresponding user based on the comparison results.

In a preferred embodiment of the present embodiment, the method for establishing a machine learning-based prognosis prediction model specifically comprises the following steps:
identifying and obtaining multiple users who have implemented a natural entity intervention from multiple medical health information systems;
obtaining the digital representation of the carbon-based biochemical data of the human body and the digital representation of the natural entity intervention data from the individual digital twin models of each user before the implementation of the corresponding natural entity intervention, as well as obtaining the digital representation of the carbon-based biochemical data of the human body and the digital representation of the natural entity intervention data after the implementation of the corresponding natural entity intervention; establishing an impact label based on the data before and after the implementation of the natural entity intervention, wherein the impact label indicates the effect of the corresponding natural entity intervention on the corresponding user;
establishing a recurrent neural network model, using the digital representation of the carbon-based biochemical data of the human body and the digital representation of the natural entity intervention data before the implementation of the corresponding natural entity intervention as input, and the corresponding impact label as output, iteratively training the recurrent neural network model to obtain the prognosis prediction model.

In a preferred embodiment of the present embodiment, the step of using the machine learning module to assist in optimizing medical health decision-making based on the digital representation of the perceptual and cognitive data of the human body and the digital representation of natural entity intervention data, and obtaining a digital representation of optimized medical health decisions matching the user, specifically comprises the following steps:
identifying and obtaining multiple users who have implemented a natural entity intervention from multiple medical health information systems;
obtaining the digital representation of the perceptual and cognitive data of the human body and the digital representation of the natural entity intervention data before the implementation of the corresponding natural entity intervention from the individual digital twin models of each user, as well as obtaining the digital representation of the carbon-based biochemical data of the human body and the digital representation of the natural entity intervention data after the implementation of the corresponding natural entity intervention; establishing an evaluation label based on the data before and after the implementation of the natural entity intervention, wherein the evaluation label indicates the corresponding user's assessment of the corresponding natural entity intervention;
establishing a recurrent neural network model; using the digital representation of the perceptual and cognitive data of the human body and the digital representation of the natural entity intervention data before the implementation of the corresponding natural entity intervention as input, and the corresponding evaluation label as output, iteratively training the recurrent neural network model to obtain the decision optimization model;
performing holistic diagnosis using the decision optimization model to obtain a digital representation of optimized medical health decisions matching the user.

In a preferred embodiment of the present embodiment, the step of receiving health data of individual users from multiple medical health information systems further comprises:
receiving multiple raw data from multiple medical health information systems for the individual user; calculating at least one new enriched data based on the relationships among the multiple raw data; placing the multiple raw data and the calculated enriched data into the corresponding user's health data. For example, based on the user's illness data, healthcare service data, and physician prescription records, and one or more relationships among the aforementioned three types of data, determining and creating at least one new enriched dataset or more relationships, and placing them into the corresponding user's health data.

Embodiment 2: This embodiment provides an electronic device, comprising a memory, a processor, and a computer program stored on the memory and executable on the processor, wherein the processor, when executing the program, implements the method for establishing a digital twin based on medical health according to any embodiment of the present invention.

Embodiment 3: This embodiment provides a computer-readable storage medium on which a computer program is stored, and when the program is executed by a processor, it implements the method for establishing a digital twin based on medical health according to any embodiment of the present invention.

Embodiment 4: Users typically lack the professional ability to address health issues using a digital twin model and necessarily require direct or indirect assistance from a service system. Therefore, this embodiment provides a health management service system for the entire life cycle, which builds a bridge between users and the digital twin model based on users' needs for decision-support data in health management. The “service” is a decision-support system for the entire life cycle of the digital twin, used by users for medical care outside of emergencies.

Specifically, the system includes a collaborative management module and the individual digital twin model of the user, wherein the individual digital twin model of the user is established by the method for establishing a digital twin based on medical health according to any embodiment of the present invention.

The collaborative management module obtains data from the individual digital twin model of the user and provides services to the user based on the obtained data. The services include a closed loop of monitoring and analysis, diagnosis prediction, prognosis prediction, intervention decision-making, and post-effect evaluation.

The data is iteratively optimized to refine the individual digital twin model, with the collaborative management module producing decision-support data through clinical preventive operations by a multidisciplinary medical team. The entangled feedback of the data provides dynamic “historical data” support for clinical preventive operations, forming a cycle of repeated iterations that optimize the model to better fit the user, making the implementation of intervention measures more precise and empowering individuals or their guardians with medical health decision-making capabilities. This creates a model of “basic individual digital twin model+X,” where the basic individual digital twin model meets the needs of daily healthcare and routine disease treatment for the entire population, and X represents advanced technological interventions. The model maps the physical and mental condition in real time and carries individualized solutions and intervention recommendations, which users can access and utilize at any time through the internet, realizing full life-cycle health management.

The above description is merely an embodiment of the present invention and does not limit the scope of the patent. Any equivalent structure or equivalent process transformation made by utilizing the content of the present invention's specification and drawings, or any direct or indirect application in other related technical fields, shall be included within the scope of patent protection of the present invention.

1. A method for establishing a digital twin based on medical health, characterized by comprising the following steps:
receiving health data of individual users from multiple medical health information systems;
generating an individual digital twin model based on the health data of individual users, wherein the individual digital twin model includes a digital representation of carbon-based biochemical data of the human body, a digital representation of perceptual and cognitive data of the human body, and a digital representation of natural entity intervention data; the digital representation of carbon-based biochemical data of the human body characterizes the user's physical physiological features; the digital representation of perceptual and cognitive data of the human body characterizes the user's psychological perception and cognitive features; the digital representation of natural entity intervention data characterizes the features of external entity exposure and intervention; and
using a machine learning module in the individual digital twin model to perform holistic diagnosis based on the digital representation of carbon-based biochemical data of the human body and the digital representation of perceptual and cognitive data of the human body, obtaining a digital representation of the user's diagnostic data; using the machine learning module in the individual digital twin model to dynamically match natural entity interventions based on the digital representation of carbon-based biochemical data of the human body and the digital representation of natural entity intervention data, obtaining a digital representation of the effects of natural entity interventions matching the user; using the machine learning module in the individual digital twin model to assist in optimizing medical health decision-making based on the digital representation of perceptual and cognitive data of the human body and the digital representation of natural entity intervention data, obtaining a digital representation of optimized medical health decisions matching the user.

2. The method for establishing a digital twin based on medical health according to claim 1, characterized in that the digital representation of carbon-based biochemical data of the human body includes a digital representation of the microstructure of the body and a digital representation of macroscopic signs; the digital representation of perceptual and cognitive data of the human body includes at least a digital representation of psychological emotions, a digital representation of habits and preferences, and a digital representation of value orientation; the digital representation of the effects of natural entity interventions includes at least a digital representation of diet, a digital representation of activities, a digital representation of geographical environment, a digital representation of medical product usage, a digital representation of medical health services systems, and a digital representation of data collected by monitoring devices.

3. The method for establishing a digital twin based on medical health according to claim 1, characterized in that the step of using the machine learning module to perform holistic diagnosis based on the digital representation of carbon-based biochemical data of the human body and the digital representation of perceptual and cognitive data of the human body to obtain a digital representation of the user's diagnostic data specifically comprises the following steps:
obtaining the digital representation of carbon-based biochemical data of the human body and the digital representation of perceptual and cognitive data of the human body from the individual digital twin models of multiple users with health issues; generating a feature data matrix based on the extracted data;
establishing a recurrent neural network model, inputting the generated feature data matrix as input, and using the corresponding user's health issues as output, iteratively training the recurrent neural network model to obtain a diagnostic model;
performing holistic diagnosis using the diagnostic model, obtaining a digital representation of the corresponding user's diagnostic data.

4. The method for establishing a digital twin based on medical health according to claim 1, characterized in that the step of using the machine learning module to dynamically match natural entity interventions based on the digital representation of carbon-based biochemical data of the human body and the digital representation of natural entity intervention data, and obtaining a digital representation of the effects of natural entity interventions matching the user specifically comprises the following steps:
establishing a machine learning-based prognosis prediction model, using the prognosis prediction model and the individual digital twin model to simulate the impact on the corresponding user after implementing various natural entity interventions;
comparing the various natural entity interventions based on the impact on the corresponding user after implementation, and dynamically matching natural entity interventions to the corresponding user based on the comparison results.

5. The method for establishing a digital twin based on medical health according to claim 4, characterized in that the method of establishing the machine learning-based prognosis prediction model specifically comprises:
identifying and obtaining multiple users who have implemented a natural entity intervention from multiple medical health information systems;
obtaining the digital representation of carbon-based biochemical data of the human body and the digital representation of natural entity intervention data before the implementation of the corresponding natural entity intervention, as well as obtaining the digital representation of carbon-based biochemical data of the human body and the digital representation of natural entity intervention data after the implementation of the corresponding natural entity intervention;
establishing an impact label based on the data before and after the implementation of the natural entity intervention, wherein the impact label indicates the effect of the corresponding natural entity intervention on the corresponding user;
establishing a recurrent neural network model, using the digital representation of carbon-based biochemical data of the human body and the digital representation of natural entity intervention data before the implementation of the corresponding natural entity intervention as input, and the corresponding impact label as output, iteratively training the recurrent neural network model to obtain the prognosis prediction model.

6. The method for establishing a digital twin based on medical health according to claim 1, characterized in that the method of establishing the machine learning-based prognosis prediction model specifically comprises:
identifying and obtaining multiple users who have implemented a natural entity intervention from multiple medical health information systems;
obtaining the digital representation of carbon-based biochemical data of the human body and the digital representation of natural entity intervention data before the implementation of the corresponding natural entity intervention, as well as obtaining the digital representation of carbon-based biochemical data of the human body and the digital representation of natural entity intervention data after the implementation of the corresponding natural entity intervention; establishing an impact label based on the data before and after the implementation of the natural entity intervention, wherein the impact label indicates the effect of the corresponding natural entity intervention on the corresponding user;
establishing a recurrent neural network model, using the digital representation of carbon-based biochemical data of the human body and the digital representation of natural entity intervention data before the implementation of the corresponding natural entity intervention as input, and the corresponding impact label as output, iteratively training the recurrent neural network model to obtain the prognosis prediction model.

7. The method for establishing a digital twin based on medical health according to claim 1, characterized in that the step of receiving health data of individual users from multiple medical health information systems further comprises:
receiving multiple raw data from multiple medical health information systems for the individual user, calculating at least one new enriched data based on the relationship among the multiple raw data; placing the multiple raw data and the calculated enriched data into the corresponding user's health data.

8. A computer-readable storage medium, characterized in that a computer program is stored on the medium, and when the program is executed by a processor, it implements the method for establishing a digital twin based on medical health according to claim 1.

9. A health management service system for the entire life cycle, characterized by comprising a collaborative management module and the individual digital twin model of the user, wherein the individual digital twin model of the user is established by the method for establishing a digital twin based on medical health according to claim 1;
the collaborative management module obtains data from the individual digital twin model of the user and provides services to the user based on the obtained data.
